(19) United States
(12) Patent Application Publication
(10) Pub. No.: US 2014/0135650 A1
(43) Pub. Date: May 15, 2014

(54) Sperm Collecting Device

(71) Applicant: LOVER HEALTH SCIENCE AND TECHNOLOGY CO., LTD., Zhejiang (CN)

(72) Inventor: Wei WU, Zhejiang (CN)

(73) Assignee: LOVER HEALTH SCIENCE AND TECHNOLOGY CO., LTD., Zhejiang (CN)

(21) Appl. No.: 13/729,019

(22) Filed: Dec. 27, 2012

(30) Foreign Application Priority Data
Nov. 12, 2012 (CN) 201220592762.1

(51) Int. Cl. A61B 10/00

(52) U.S. Cl. CPC A61B 10/0058
USPC 600/573

(57) ABSTRACT

A sperm collecting device is provided. The sperm collecting device includes a main body, a cap that is separately connected to a first end of the main body, a hanger that is rotatably connected to a second end of the main body and a soft body that is disposed in the main body and having a penis passage. The sperm collecting device further includes a pressing unit. The pressing unit includes a pressing element and a pressing plate, wherein the pressing element is disposed at the main body, and the main body has a pressing opening disposed corresponding to a pressing portion of the pressing element. The pressing plate is disposed between the main body and the soft body corresponding to the pressing opening.

### CROSS-REFERENCE TO RELATED APPLICATIONS

This Non-provisional application claims priority under 35 U.S.C. §119(a) on Patent Application No(s). 201220592762.1 filed in People's Republic of China, the entire contents of which are hereby incorporated by reference.

### BACKGROUND OF THE INVENTION

### Field of the Invention

This invention relates to a medical apparatus and, more particular, to a sperm collecting device.

Collecting sperms is necessary for the diagnosis of many diseases of male reproductive systems. Clinically, the sperms of a patient are mainly collected by means of masturbation, which is not conductive to quick, convenient, and aseptic sperm collection. It may also have undesired effects on the diagnosis and examination, such as incorrect results in examination. At present, sperm collecting devices available in the market are simple in structure, and the main bodies thereof are of certain hardness. Those devices can not achieve a squeezing function and can not provide a sufficient stimulation on a penis. Furthermore, the devices do not have additional vibration functions and are not easy to excite users during use.

The disclosed sperm collecting devices are directed at solving one or more problems set forth above and other problems.

### BRIEF SUMMARY OF THE INVENTION

To improve the prior art, this invention provides a sperm collecting device with a massaging function and a better massaging effect.

One aspect of the present disclosure provides a sperm collecting device. The sperm collecting device includes a main body, a cap that is separately connected to a first end of the main body, a hanger that is rotatably connected to a second end of the main body and a soft body that is disposed in the main body and having a penis passage. The sperm collecting device further includes a pressing unit. The pressing unit includes a pressing element and a pressing plate, wherein the pressing element is disposed at the main body, and the main body has a pressing opening disposed corresponding to a pressing portion of the pressing element. The pressing plate is disposed between the main body and the soft body corresponding to the pressing opening.

In one embodiment of the invention, the main body may include a large cylindrical body and a small cylindrical body. A first end of the large cylindrical body is separately connected to the cap, and a second end of the large cylindrical body is connected to the small cylindrical body.

In one embodiment of the invention, the pressing element may be substantially cylindrical in shape, and the pressing element may be sleeved on the outside of the main body or sleeved between the pressing plate and the main body.

In one embodiment of the invention, the pressing element may be inlaid in the pressing opening.

In one embodiment of the invention, the pressing element may include a vibration unit disposed at the main body.

In one embodiment of the invention, the vibration unit may further include a vibration motor, a motor sleeve, a circuit board, and a press button. The motor sleeve is sleeved on the inner side of the main body. The vibration motor is attached to the motor sleeve and electrically connected to the circuit board. The circuit board and the press button are disposed at the main body, and the press button is electrically connected to the circuit board.

In one embodiment of the invention, the sperm collecting device may further include a power source and a power connecting member. The power source and the power connecting member are disposed at the end of the main body close to the hanger, and the power source is electrically connected to the circuit board and the power connecting member.

In one embodiment of the invention, the end of the main body close to the hanger may have at least one opening, and the hanger may have at least one protrudent portion correspondingly. The hanger is rotatably connected to the main body via the mutual fastening of the protrudent portion and the opening.

In one embodiment of the invention, the shape of the pressing plate may be matched with the profile of the soft body, and the length of the pressing plate along the longitudinal direction of the main body is longer than that of the pressing opening along the longitudinal direction of the main body.

In one embodiment of the invention, one end of the pressing plate may have a protrusion for attachment, and the pressing plate may be attached to the main body via the protrusion.

In one embodiment of the invention, the sperm collecting device may further include a fastening ring, and the soft body may be attached to the main body via the fastening ring.

To sum up, via the pressing unit disposed at the main body, the sperm collecting device in the invention can achieve the squeezing function and strengthen stimulation on a penis, such that a user can get excited more easily to facilitate collecting sperm. By the pressing plate, the penis moving in the penis passage suffers more uniform pressure and the pressure area is enlarged thus to achieve the better squeezing effect, greatly improving the comfortable sensation and pleasant sensation of the user during sperm collection. Meanwhile, the speed of the sperm collection is accelerated, and the efficiency of the sperm collection is enhanced. In addition, via the hanger, the sperm collecting device in the invention can be hung for storage after use, which is healthier and more convenient, and the device can also be hung for display on sale to make it more remarkable. The hanger in the invention is designed to be rotatable and can be rotated and received when not used thus to save space.

Furthermore, since the opening is disposed at the end of the main body close to the hanger, the air in the penis passage can be smoothly expelled out of the sperm collecting device thus to allow the penis to smoothly move in a reciprocating way in the penis passage, and the influence generated by air pressure difference is eliminated. Meanwhile, the opening in the invention can also function as a connecting hole. The protrudent portion of the hanger is inserted into the opening, and thus the hanger can be rotatably connected to the main body.

In addition, via the vibration motor, the sperm collecting device achieves the vibration function and strengthens the stimulation on the penis, making the user excited as soon as possible and achieving a better sperm collecting effect. With the power source and the power connecting member, the sperm collecting device can be used anytime and anywhere. Meanwhile, via the fastening ring, the soft body disposed in the main body does not drop off easily thus to achieve the fixing of the soft body. Further, it's convenient to take out and clean the soft body.

Furthermore, since the sperm collecting device in the invention can achieve an effect of close wrapping and rubbing and can make the user comfortable, the sperm collecting device in the invention can be used not only as a medical device, but also as a pleasure device. Thus, the user can quickly and comfortably console himself without a hand-job.

These and other features, aspects, and advantages of the present invention will become better understood with regard to the following description, appended claims, and accompanying drawings. Other aspects of the present disclosure can be understood by those skilled in the art in light of the description, the claims, and the drawings of the present disclosure.

### BRIEF DESCRIPTION OF THE DRAWINGS

FIG. 1 illustrates an exploded schematic diagram of an exemplary sperm collecting device consistent with the disclosed embodiments;

FIG. 2 illustrates a partial schematic diagram of an exemplary sperm collecting device consistent with the disclosed embodiments;

FIG. 3 illustrates a sectional schematic diagram of an exemplary sperm collecting device consistent with the disclosed embodiments;

FIG. 4 is a sectional diagram along the A-A direction in FIG. 3; and

FIG. 5 illustrates a schematic view of different states of an exemplary hanger of an exemplary sperm collecting device consistent with the disclosed embodiments.

### DETAILED DESCRIPTION OF THE INVENTION

Reference will now be made in detail to exemplary embodiments of the invention, which are illustrated in the accompanying drawings. Wherever possible, the same reference numbers will be used throughout the drawings to refer to the same or like parts.

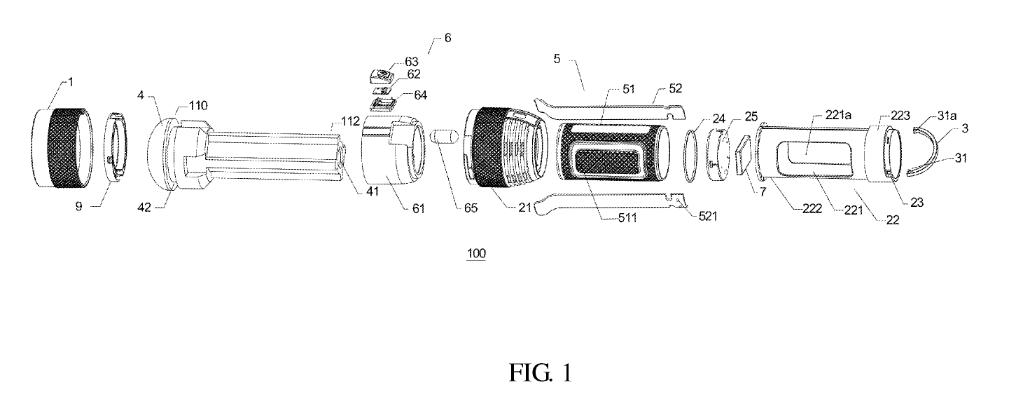

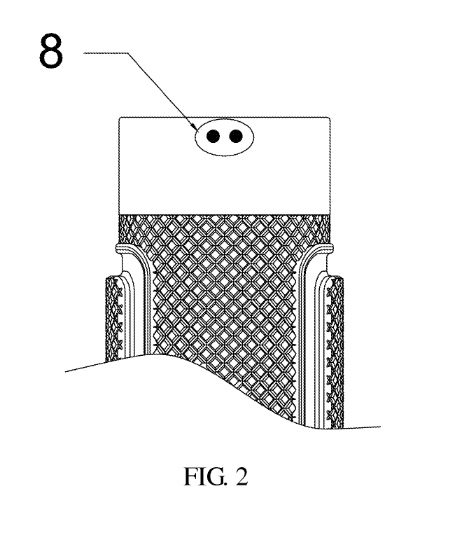

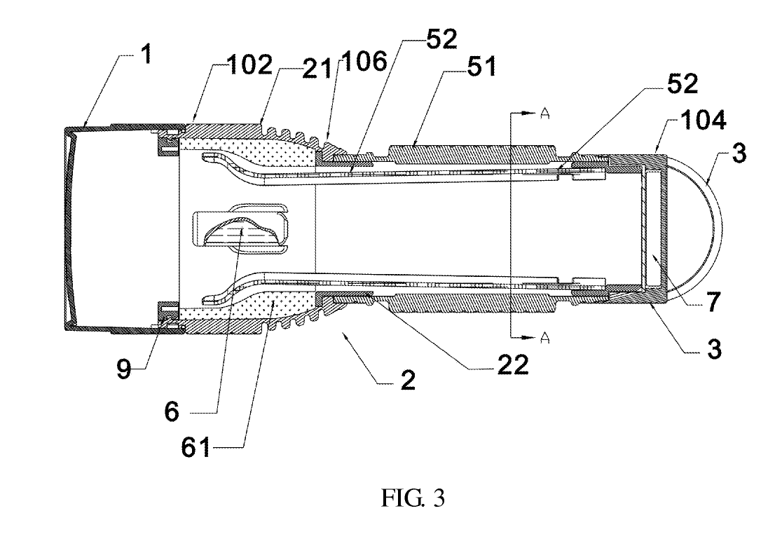

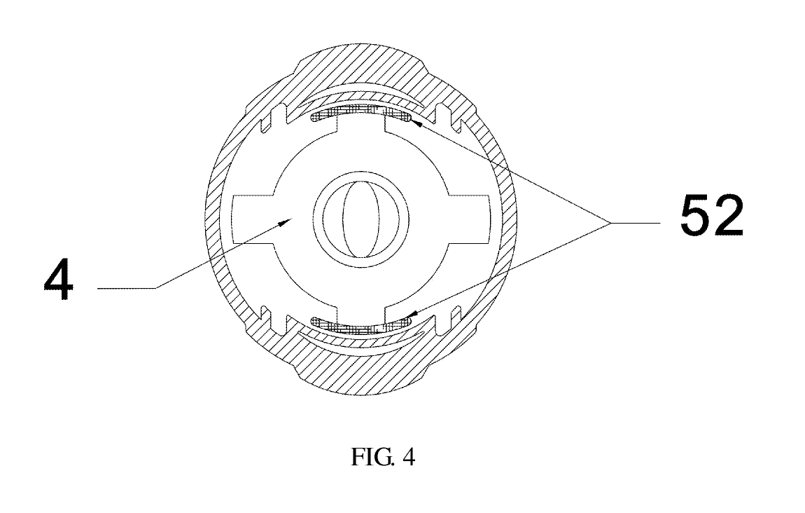

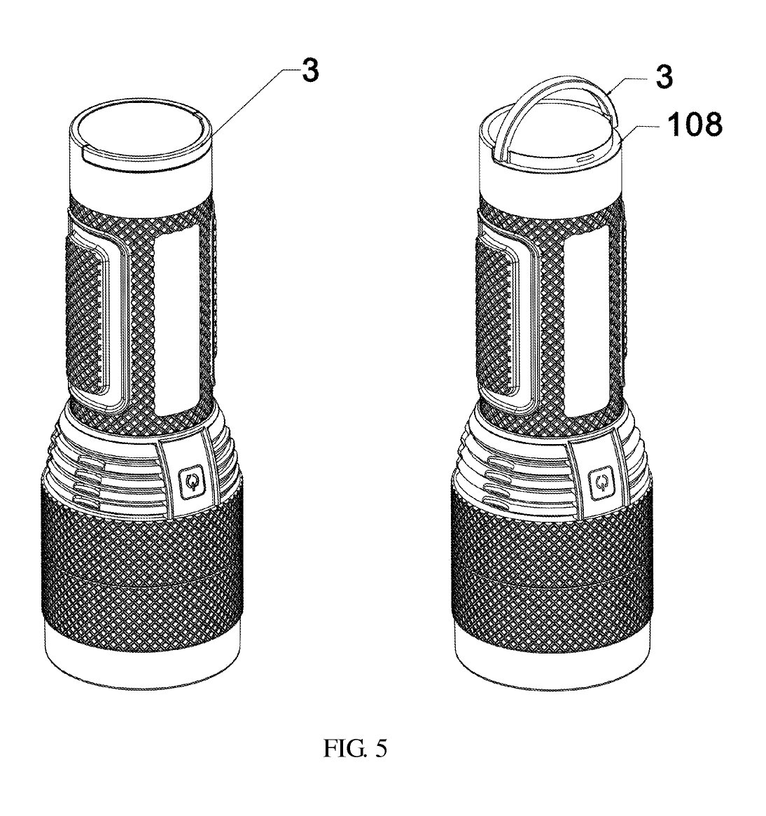

FIG. 1 illustrates an exploded schematic diagram of an exemplary sperm collecting device according to this invention. FIG. 2 illustrates a partial schematic diagram showing the sperm collecting device consistent with the disclosed embodiments. FIG. 3 illustrates a sectional schematic diagram of the sperm collecting device consistent with the disclosed embodiments. FIG. 4 illustrates a sectional diagram along the A-A direction in FIG. 3. FIG. 5 illustrates a schematic view of different states of a hanger of the sperm collecting device consistent with the disclosed embodiments. Please refer to FIG. 1 to FIG. 5 together.

As shown in the FIGS. 1-5, a sperm collecting device 100 includes a cap 1, a main body 2, a hanger 3, a soft body 4, and a pressing unit 5. The cap 1 is separately connected to a first end 102 of the main body 2. The hanger 3 is rotatably connected to a second end 104 of the main body 2. The soft body 4 is disposed in the main body 2 and has a penis passage 41. The pressing unit 5 includes a pressing element 51 and a pressing plate 52. The pressing element 51 is disposed at the main body 2, and the main body 2 has a first pressing opening 221 and a second pressing opening 221a. The pressing element 51 may be exposed through the first pressing opening 221 to allow a user to squeeze the pressing element 51. The pressing plate 52 is configured to be located between the main body 2 and the soft body 4.

As shown in FIGS. 1-5, the main body 2 includes a large cylindrical body 21 and a small cylindrical body 22. The first end 102, which is located on the large cylindrical body 21, is separately connected to the cap 1, and a second end 106 of the large cylindrical body 21 is connected to the small cylindrical body 22. The large cylindrical body 21 has a relatively larger diameter at the first end 102 and the diameter gradually reduces to allow the body 21 to be fastened, threaded, or adhered to the small cylindrical body 22 at the second end 106. As shown in FIGS. 1-5 the cap 1 is fastened to the large cylindrical body 21. When the sperm collecting device 100 is in use, the cap 1 may be pulled and separated from the large cylindrical body 21. When the sperm collecting device 100 is not in use, the cap 1 may cover and be fastened to the large cylindrical body 21.

As shown in FIGS. 1-5, the small cylindrical body 22 includes an upper portion 222 and a tail portion 223. When the device 100 is in use, the tail portion 223 is exposed to the air. The upper portion 222 and the tail portion 223 may be inseparably connected to each other. The upper portion 222 and the tail portion 223 may be integrally molded. When the upper portion 222 and the tail portion 223 are inseparably connected, the tail portion 223 may not be easy to drop after long-term use. The upper portion 222 and the tail portion 223 may also be separably connected to each other.

As shown in FIGS. 1-5, the main body 2 close to the hanger 3 has at least a first opening 23 close to the end 104, and the hanger 3 has at least a first protrudent portion 31. The first protrudent portion 31 may be fastened to the first opening 23, and the hanger 3 may be rotatably connected to the tail portion 223 of the main body 2. As shown in FIGS. 1-5, the main body 2 may have a second opening (not shown) and the hanger 2 may have a second protrudent portion 31a correspondingly. The device 100 may include additional openings and hangers and protrudent portions thereon. As shown in FIGS. 1-5, the main body 2 further includes a sealing ring 24 and a sealing element 25. The sealing ring 24 and the sealing element 25 are disposed in the tail portion 223 to prevent the collected sperms from leaking out of the sperm collecting device 100.

As shown in FIGS. 1-5, the opening 23 may also function as a connecting member. In certain embodiments, the connecting member may be different to the opening 23. The opening 23 may allow the air in the penis passage 41 to be smoothly expelled out of the sperm collecting device 100. As a result, a penis may move back and forth in the penis passage 41 smoothly. The disposition of the opening 23 avoids forming a closed space in the main body 2 during use. In cases where the closed space is formed, the air in the space will impede the penis from moving in the penis passage 41 toward the side close to the hanger 3, causing the reciprocating movement of the penis is unsmooth.

As shown FIGS. 1-5, the hanger 3 is semicircular in shape, and the end 104 has a semicircular recess 108 for receiving the hanger 3. The hanger 3 may be received in the recess such that the bottom of the end 104 is a flat surface. The hanger 3 may be rotated for certain degrees between 1 to 90 degrees for convenient use. Via the hanger 3, the sperm collecting device 100 may be hung for storage when not in use, and the device may also be hung for display as an item for purchase. The hanger 3 in the invention is designed to be rotatable and can be rotated and received when not used thus to save space.

As shown in FIGS. 1-5, the pressing openings 221 and 221a are symmetrically disposed at the upper portion 222 of the small cylindrical body 22. The pressing openings 221 and 221a may be in a shape of rectangle, with a length of about 30-120 mm and a width of about 20-80 mm. In certain embodiments, the length of the pressing opening is about 100 mm and the width is about 50 mm. However, the invention is not limited thereto. In other embodiments, the pressing openings may be in any other shape, such as an elliptic shape.

As shown in FIGS. 1-5, the sperm collecting device 100 may further include a vibration unit 6 disposed at the main body 2. The vibration unit 6 includes a vibration motor 65, a motor sleeve 61, a circuit board 62 and a press button 63. The motor sleeve 61 may be sleeved on the inner side of the main body 2. The motor sleeve 61 may be sleeved on the inner side of the large cylindrical body 21. The motor sleeve 61 may be also located in other positions. The vibration motor 65 is attached to the motor sleeve 61 and electrically connected to the circuit board 62. The motor sleeve 61 may have a motor attaching element to attach the vibration motor 65. The circuit board 62 and the press button 63 are disposed on the main body 2, and the press button 63 is electrically connected to the circuit board 62.

As shown in FIGS. 1-5, the sperm collecting device 100 may further include a circuit board fixing element 64. The circuit board 62 may be fixed on the inner wall of the large cylindrical body 21 via the circuit board fixing element 64, and the motor sleeve 61 may have a space at the corresponding position to accommodate the circuit board fixing element 64 and the circuit board 62 on the inner wall of the large cylindrical body 21. The large cylindrical body 21 may have an opening to expose the press button 63 through the opening to a user. The large cylindrical body 21 may also have no opening at the position corresponding to the press button 63. The spot on the large cylindrical body 21 corresponding to the press button 63 may be made of a material that allows a user to press on the button 63 through the spot. As shown in FIGS. 1-5, the sperm collecting device 100 may further include a power source 7. The power source 7 may be a regular disposable battery or a rechargeable battery.

As shown in FIGS. 1-5, the device 100 may further include a power connecting member 8. The power source 7 and the power connecting member 8 may be disposed at close to the end 104, and the power source 7 is electrically connected to the circuit board 62 and the power connecting member 8. The power source 7 is disposed between the sealing element 25 and the tail portion 223. When the power source 7 is rechargeable through the power connecting member 8, the sperm collecting device may be used at different locations during different time. The power source 7 may drive the vibration motor 65, to cause the sperm collecting device vibrate and strengthen stimulation on the penis, so the user may be stimulated in relatively shorter time for a better sperm collecting effect.

As shown in FIG. 1, the soft body 4 has a front end 110 and a rear end 112. The front end 110 may be protruded out from the main body 2, and the rear end 112 may direct the insertion of the soft body 4 through the large cylindrical body 21 and the small cylindrical body 22 of the main body 2. The front end 110 may be semi-spherical. When the user's penis moves back and forth in the penis passage 41, the front end 110 of the soft body 4 isolates the hard material of the main body 2 from touching the human body thus to increase comfort in use. As shown in FIGS. 1-5, the soft body 4 is shaped to be centrally symmetric. The soft body 4 has four orthogonal convex edges, and the number of the pressing plates 52 is two. When pressed, the two pressing plates 52 are directly pressed onto the two opposite convex edges.

As shown in FIG. 1, the sperm collecting device 100 may further include a fastening member 9, and the soft body 4 may be attached to the main body 2 via the fastening member 9. An attaching member (not shown in the drawing) may be disposed at the inner wall of the large cylindrical body 21. A recess 42 may be disposed at the soft body 4. The fastening member 9 may be attached to the recess 42, and then the attaching member may be attached to the fastening member 9. Via the fastening member 9, the soft body 4 may be attached to the main body 2 in a relatively stable manner. Further, the fastening member 9 and the attaching member may facilitate the removal of the soft body 4 from the main body 2 for cleaning and other purposes.

As shown in FIGS. 1-5, the pressing element 51 is in a cylindrical shape, and the pressing element 51 is sleeved on the outside of the main body 2. The pressing element 51 may also be in other shape. In certain embodiments, the pressing element 51 may be sleeved on the inside of the main body 2 and located between the pressing plate 51 and the main body 2. In certain embodiments, the pressing element 51 can be inlaid in the pressing opening 221.

A part of the pressing element 51 may be made of relatively hard material. As shown in FIGS. 1-5, a pressing portion 511 of the pressing element 51, which does not cover the entire surface of the pressing element 51, is made of relatively hard material, while the rest part of the pressing element 51 is made of soft material, such as rubber or silicone. Therefore, when the pressing portion 511 is pressed, the soft material on the pressing element 51 is stretched towards the inside of the pressing element 51, such that the pressing portion 511 moves towards the inside to generate a massaging effect. The objective of making the pressing portion 511 by relatively hard material is to reduce loss of pressing force during transmission of the pressing force. In certain embodiments, the pressing element 51 may be made entirely of soft material.

When the sperm collecting device 100 is used, the user presses the pressing element 51, and a part of the pressing element 51 is elastically deformed and is bent towards the inside of the main body 2. The pressing portion 511 passes through the pressing opening 221 until reaching the pressing plate 52 so as to transmit the pressure to the pressing plate 52. Subsequently, the pressing plate 52 presses the soft body 4 to compress the penis passage 41 disposed in the soft body 4, such that the penis in the penis passage 41 may be pressed upon. Via the pressing plate 52, the penis moving in the penis passage 41 may receive relatively uniform around the penis and a relatively larger area may receive the pressure to achieve a better effect of massaging the penis and to improve the comfort and pleasantness of the user during sperm collection. Meanwhile, the speed of the sperm collection may be accelerated, and the efficiency of the sperm collection may be enhanced.

Without the pressing plate 52, when the user exerts the pressure onto the pressing element 51, only the region on the soft body 4 corresponding to the pressing fingers receives the pressure (the maximal size of the pressure area can only be up to the size of the pressing portion 511). In other words, without the pressing plate 52, only a part of the penis passage 41 is pressed. As shown in FIGS. 1-5, one end of the pressing plate 52 has a protrusion 521, and the pressing plate 52 may be attached to the small cylindrical body 22 via the protrusion 521.

As shown in FIGS. 1-5, the pressing plate 52 is configured to match with the profile of the soft body 4, and the length of the pressing plate 52 along the longitudinal direction of the main body 2 is longer than that of the pressing opening 221 along the longitudinal direction of the main body. The longitudinal length of the pressing plate 52 may be slightly shorter than that of the penis passage 41. In other words, once the user exerts the pressure, the pressing plate 52 will press the entire penis passage 41 to massage the entire penis within the penis passage 41 so as to simulate the effect of intercourse with a human being. The pressing plate 52 may take other shape and size.

To sum up, via the pressing unit 5 disposed at the main body 2, the sperm collecting device 100 consistent with the disclosed embodiment can achieve the massaging effect and enhance stimulation on the penis, such that the user may be excited with relative easiness to facilitate collecting the sperms. By the pressing plate 52, the penis moving in the penis passage receives relatively uniform pressure around the surface of the penis. Therefore, a better massaging effect may be achieved, to improve the comfort and pleasantness of the user during sperm collection. Meanwhile, the speed of the sperm collection is accelerated, and the efficiency of the sperm collection is enhanced. In addition, via the hanger 3, the sperm collecting device in the invention can be hung for storage after use, which is healthier and more convenient, and the device can also be hung for display on sale to make it more remarkable. The hanger 3 in the invention is designed to be rotatable and can be rotated and received when not used thus to save space.

Furthermore, since the opening 23 is disposed at the end of the main body 2 close to the hanger 3, the air in the penis passage 41 can be smoothly expelled out of the sperm collecting device thus to allow the penis to smoothly move in a reciprocating way in the penis passage 41, and the influence generated by air pressure difference is eliminated. Meanwhile, the opening 23 in the invention can also function as a connecting hole. The protrudent portion 31 of the hanger 3 is inserted into the opening 23, and thus the hanger 3 can be rotatably connected to the main body 2.

In addition, via the vibration motor 65, the sperm collecting device achieves the vibration function and strengthens the stimulation on the penis, making the user excited as soon as possible and achieving a better sperm collecting effect. With the power source 7 and the power connecting member 8, the sperm collecting device can be used anytime and anywhere. Meanwhile, via the fastening ring 9, the soft body 4 disposed in the main body 2 does not drop off easily thus to achieve the fixing of the soft body 4. Further, it's convenient to take out and clean the soft body 4.

Furthermore, since the sperm collecting device in the invention can achieve the effect of close wrapping and rubbing and can make the user comfortable, the sperm collecting device in the invention can be used not only as a medical device, but also as a pleasure device. Thus, the user can quickly and comfortably console himself without a hand-job. While various embodiments in accordance with the present invention have been shown and described, it is understood that the invention is not limited thereto. The present invention may be changed, modified and further applied by those skilled in the art. Therefore, this invention is not limited to the detail shown and described previously, but also includes all such changes and modifications.

For example, the penis passage 41 may be made in different sizes to adjust the need of different persons.

What is claimed is:

1. A sperm collecting device, comprising:
a main body;
a cap, separately connected to a first end of the main body;
a hanger, rotatably connected to a second end of the main body;
a soft body, disposed in the main body and having a penis passage; and
a pressing unit, including a pressing element and a pressing plate, wherein the pressing element is disposed at the main body, the main body has a pressing opening disposed corresponding to a pressing portion of the pressing element, and corresponding to the pressing opening, the pressing plate is disposed between the main body and the soft body.

2. The sperm collecting device according to claim 1, wherein the main body comprises a large cylindrical body and a small cylindrical body, a first end of the large cylindrical body is separately connected to the cap, and a second end of the large cylindrical body is connected to the small cylindrical body.

3. The sperm collecting device according to claim 1, wherein the pressing element is substantially cylindrical in shape, and the pressing element is sleeved on the outside of the main body or sleeved between the pressing plate and the main body.

4. The sperm collecting device according to claim 1, wherein the pressing element is inlaid in the pressing opening.

5. The sperm collecting device according to claim 1, further comprising a vibration unit disposed at the main body.

6. The sperm collecting device according to claim 5, the vibration unit comprising a vibration motor, a motor sleeve, a circuit board, and a press button, wherein the motor sleeve is sleeved on the inner side of the main body, the vibration motor is attached to the motor sleeve and electrically connected to the circuit board, the circuit board and the press button are disposed at the main body, and the press button is electrically connected to the circuit board.

7. The sperm collecting device according to claim 6, further comprising a power source and a power connecting member, wherein the power source and the power connecting member are disposed at the end of the main body close to the hanger, and the power source is electrically connected to the circuit board and the power connecting member.

8. The sperm collecting device according to claim 1, wherein the end of the main body close to the hanger has at least one opening, the hanger has at least one protrudent portion correspondingly, and the hanger is rotatably connected to the main body via the mutual fastening of the protrudent portion and the opening.

9. The sperm collecting device according to claim 1, wherein the shape of the pressing plate is matched with the profile of the soft body, and the length of the pressing plate along the longitudinal direction of the main body is longer than that of the pressing opening along the longitudinal direction of the main body.

10. The sperm collecting device according to claim 1, wherein one end of the pressing plate has a protrusion for attachment, and the pressing plate is attached to the main body via the protrusion.

11. The sperm collecting device according to claim 1, further comprising a fastening ring, wherein the soft body is attached to the main body via the fastening ring.
